(19) United States
(12) Patent Application Publication
(10) Pub. No.: US 2001/0007926 A1
(43) Pub. Date: Jul. 12, 2001

(54) Biological sample collection kit

(76) Inventor: David Trudil, Riesterstown, MD (US)

Correspondence Address: Jonathan E. Grant, Suite 210, 2120 L Street, N.W., Washington, DC, 20037, US

(21) Appl. No.: 09/748,216

(22) Filed: Dec. 27, 2000

Related U.S. Application Data
(63) Continuation-in-part of application No. 09/160,153, filed on Sep. 25, 1998, now Pat. No. 6,176,836.

(51) Int. Cl. A61B 10/00

(52) U.S. Cl. 600/573; 206/569; 206/363

(57) ABSTRACT

The present invention discloses a biological sample collection kit which can be utilized to collect samples of biological origin for use in any recovery and identification method.

The biological sample collection kit comprises a sterile device for collecting a sample, a device for depositing and holding the sample, a means for transferring the sample, and means for storing the storing the sample to be tested. There may also be means for identifying the sample and for diluting the sample. The biological sample collection kit preferably contains an outer bag which holds, in a sterile environment, all of the items used in the testing procedures. Additionally, a container of collection fluid is preferably included in the sample collection kit.

The following application is a continuation-in-part of 09/160,153 filed Sep. 25, 2000.

### BACKGROUND OF THE INVENTION

1. Field of the Invention

The present invention discloses a biological sample collection kit allowing for the collection and safe storing of samples.

2. Description of the Prior Art

Collection of microscopic biological samples has been random. For example, when a technician wants to test a surface for biological or chemical agents, he or she frequently has to search for a sterile cotton swab or cloth, wet it down if the surface is dry, and then wipe the surface. From there, a means must be found to transfer the absorbed material from the collection device (i.e., the cotton swab or cloth), to a storage place or an assay.

By not having all of the materials in one package which are necessary for collecting and testing the sample in one package, the risk of contamination of the sample and getting erroneous results is increased. Additionally, a great deal of time is wasted when the technician has to look around to find all of the collection and storage devices needed to safely collect and store a sample.

In the field, outside of the laboratory, it is even more necessary to have an efficient, light weight means of collecting and storing samples for testing in order to obtain more accurate results in a short period of time. A collection kit in the field must be self-contained as in the field, there is no lab supply cabinets or supply companies upon which to rely.

### SUMMARY OF THE INVENTION

The present invention discloses a biological sample collection kit which can be utilized to collect samples of biological origin for use in any recovery and identification method. The identification method may be PCR, luminescence, immunological, flow cytometry, etc.

The biological sample collection kit comprises a sterile means for collecting a sample, a means for depositing and holding the sample, a means for transferring the sample, and means for storing the storing the sample to be tested. There may also be means for identifying the sample and for diluting the sample. The biological sample collection kit preferably contains an outer bag which holds, in a sterile environment, all of the items used in the testing procedures. Additionally, a container of collection fluid is preferably included in the sample collection kit.

### BRIEF DESCRIPTION OF THE DRAWINGS

FIG. 1 is a perspective view of the storage bag;

FIG. 2 is a perspective view of a collection fluid container;

FIG. 3 is a perspective view of a sample collection container;

FIG. 4 is a perspective view of a sterile pipette;

FIG. 5 is a perspective view of a filtered bag;

FIG. 6 is a perspective view of a sponge;

FIG. 7 is a perspective view of a swab;

FIG. 8 is a perspective view of a cotton swab;

FIG. 9 is a perspective view of a syringe;

FIG. 10 is a perspective view of a sterile syringe filter;

FIG. 11 is a perspective view of a slide lock bag;

FIG. 12 is a perspective view of a collection tube;

FIG. 13 is a perspective view of a bio-hazard bag;

FIG. 14 is a perspective view of a sterile collection bag;

FIG. 15 is a perspective view of a forceps;

FIG. 16 is a perspective view of a spatula; and

FIG. 17 is a perspective view of a sterile glove.

### DETAILED DESCRIPTION OF THE INVENTION

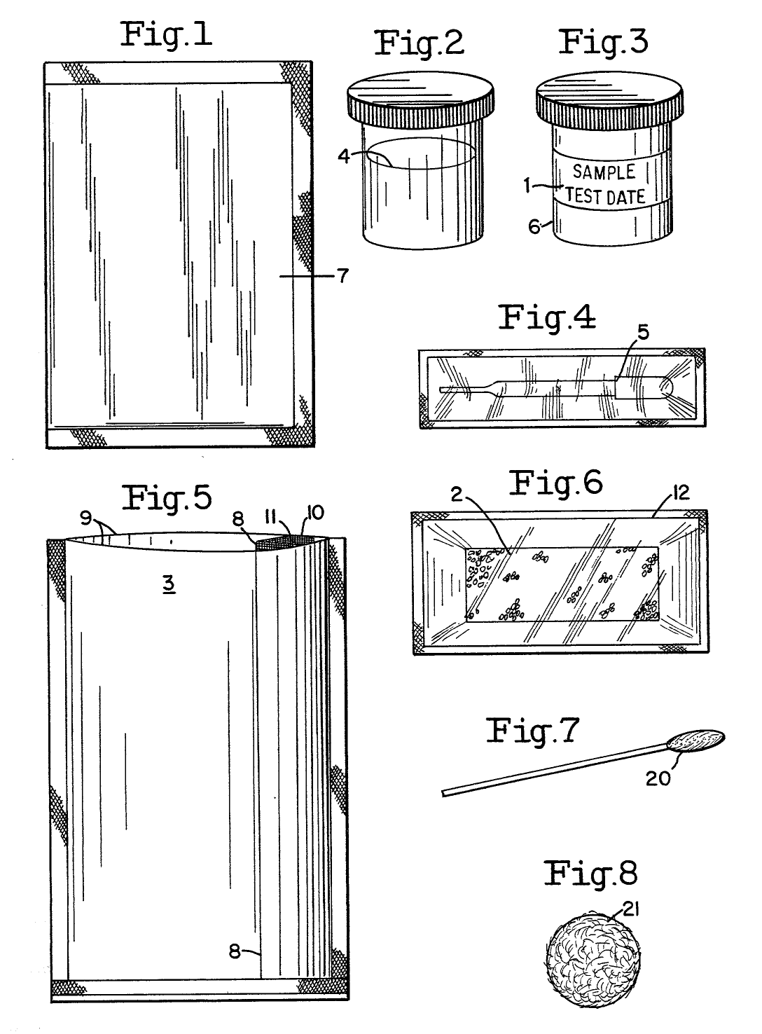

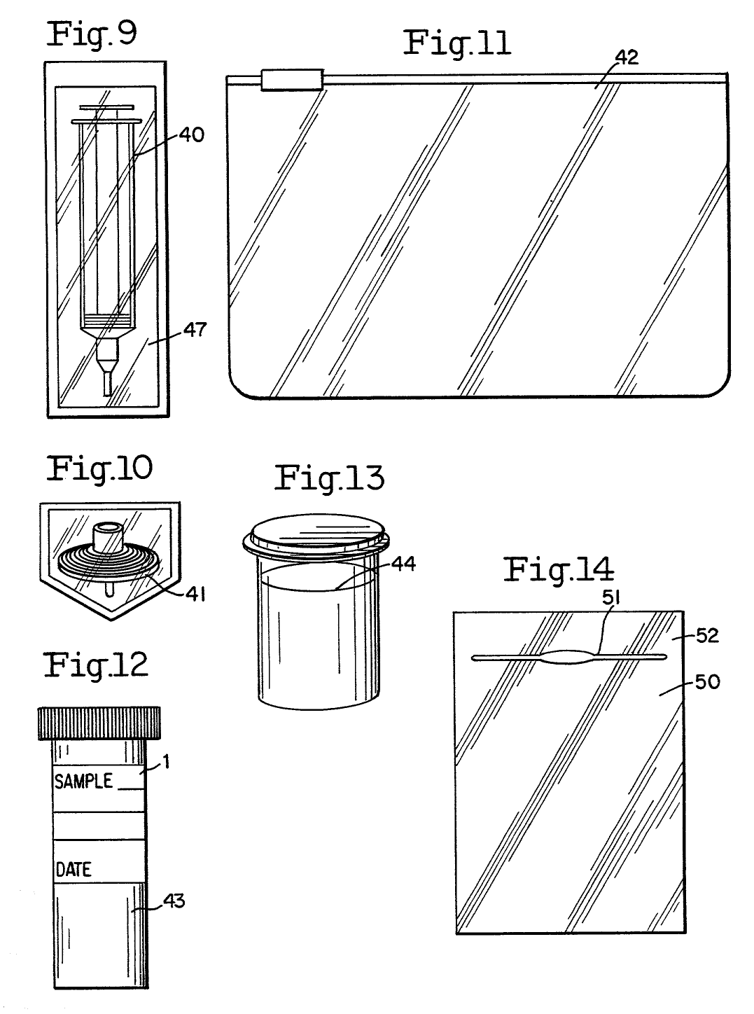

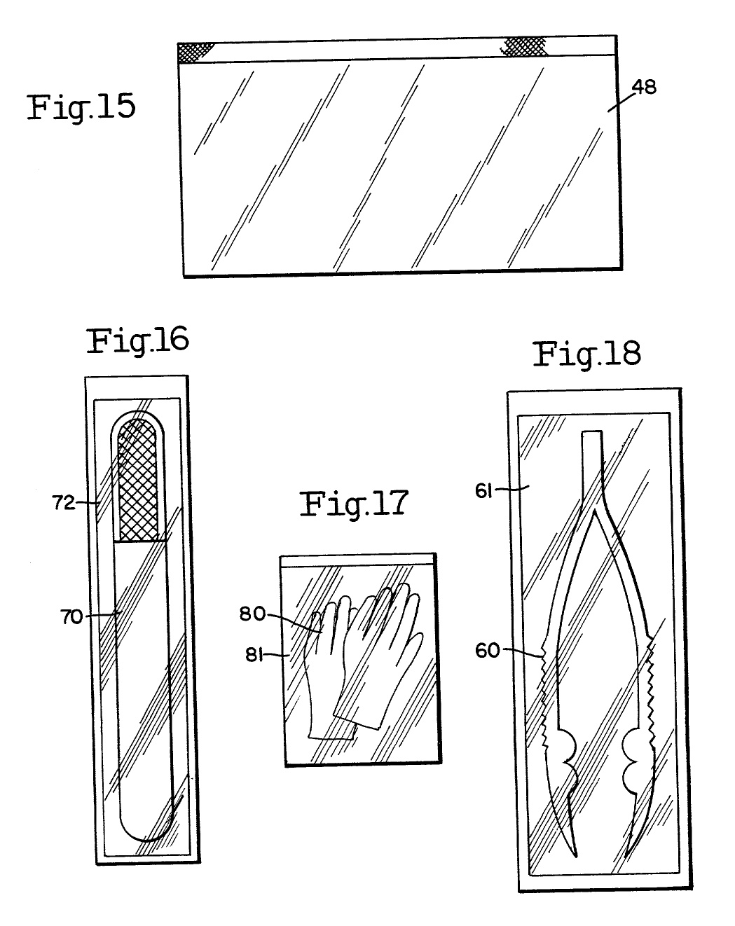

Referring to FIGS. 1-16, the sample collection kit preferably contains a sponge 2, a filtered bag 3, a buffer solution 4, a pipette 5, and a specimen collection vile 6. In a preferred embodiment of the invention, these items are kept in a collection kit bag 7. The collection kit bag 7 is preferably a sealed bag, which is preferably hermetically sealed, and sterile inside the bag. The bag may be made out of a metal foil, or a plastic.

The sponge 2 is preferably sterile, and kept in a bag 12, preferably a stomacher bag The sponge 2 will be removed from the bag 12 just prior to use. The sterile sponge 2 is used to wipe surfaces to be tested for bacteria, chemicals or other foreign matter from a surface area. If the area to be tested is dry, the sponge 2 may be first hydrated with the buffer solution or asceptic collection fluid 4. The buffer solution may be a carbonate, phosphate, or any other appropriate buffer. The buffer may also be a sterile water, a sterile saline solution or some other like sterile solution.

In another embodiment of the invention, a sterile swab 20 is used in place of the sponge. Similarly, a sterile cotton ball 21 may be used, or any other absorbent material.

The filtered bag 3 is preferably a stomacher bag 3, with the bag having a filter 8 within said bag. This filter 8 may be a porous plastic or a paper filter, adhered to the walls 9 of the bag 3, with an opening 10 at the top of the bag, and preferably cylindrical in shape. The filter 8 preferably runs the length of the bag, with the filter 8 being attached to the front and back of the bag.

In place of the bag, any sterile receptacle may be used. However, a plastic sterile bag, with a filter and a stomacher bag, is preferred.

After the sponge 2 is used to wipe down a surface area to be tested, it is placed in a general part of the filtered bag 3. After the sponge is placed in the filter bag 3, it is squeezed through the filter bag 3. That is, the user squeezes the filter bag 3 with the sponge 2 inside the filter bag 3. If so desired, additional buffer solution 4 may be added to the general part of the filtered bag 3. It is best that the filtered bag be gently shaken to mix the contents.

A pipette 5 or syringe, preferably a sterile transfer pipette contained in a sealed container 12 until use, is inserted into the opening of the filter compartment 11. The filter serves the purpose of keeping out any large, extraneous material from the fluid to be tested, but which the sponge 2 may have picked up from the testing area, including paint chips, dust, wood chips, etc.

The sample is drawn up into the pipette 5. From there, the sample may be placed in a collection tube 13, or the sample may be directly assayed. Labels 1 included in the kit may be attached to the collection tube properly identify the sample.

In another embodiment of the invention, the collection kit includes, instead of a pipette, at least one syringe 40, along with at least one sterile syringe filter 41, at least one bag 42, and preferably a slide lock bag, at least one collection receptacle 43, preferably a collection tube, and at least one bottle of aseptic collection fluid 4. The aseptic collection fluid may be sterile water or a sterile saline solution. The sterile collection tubes may have labels 1 attached, or may have labels included to be attached to the sterile collection tubes. It is preferred that there be at least two syringes 40, two collection tubes 43, and two syringe collection filters 41. After the aseptic fluid 4 has been dispersed on the surface to be tested, a sample may be drawn up through a syringe 40, and then, after the syringe filter 41 has been attached, the fluid may be expelled through the syringe 40, and syringe filter 41, and into the collection tubes 43. The collection tube 43 or receptacle is then sealed, identified, and placed in the slide lock bag 42. The used filters, and syringes, may also be placed in the bag, if desired.

If the sample to be tested is a liquid, the aseptic collection fluid may be added to the collection tubes after the sample has been collected. All of the items are kept in a sealed bio-hazard bag 46 until ready for use.

The syringes are also encased in a sterile wrapper 47. A Bio-hazard bag 48 is also included in the kit.

In another preferred embodiment of the invention, a liquid sample collection kit comprises a sterile whirl-pak collection bag 50, which has a wire seal 51 at the top 52 of the bag 50 running along the perimeter of the opening of the bag with extended tabs for gripping and twisting the bag closed. The collection kit also includes a slide lock bag 42, and at least one sterile syringe. The liquid sample collection kit is sealed bag 7. To use the liquid sample collection kit, the sealed bag 7 is opened, the contents are removed, and the slide lock bag is marked. The sample is collected using the syringes as necessary. The syringes with the sample are placed into the collection bag, which is then closed. The collection bag is then placed into a slide lock bag, and the slide is closed.

A collection kit for the collection of an air sample is also envisioned by this kit. Filters are normally set up on roof tops to collect air samples. In other open field situations, where rapid testing of air samples is desired, particularly in the military arena, filters are fitted in conjunction with air pumps, which can filter several liters of air in a short time period. The collection kit for the collection of an air sample comprises a sterile collection tube 43, an aseptical collection fluid 4, a slide lock bag 42, and forceps 60. To use this kit, the sealed bag 7 is opened, and the contents are removed. The sample, which is usually a filter of some sort, is transferred to the collection tube, by means of the forceps 60, which are sterile and packaged 61.. Aseptical solution may be added. The collection tube is closed, and placed into a slide lock bag. The slide is then closed.

A collection kit may also be used to collect a powder or to collect a sample from a small surface. The collection kit for collecting a powder sample or a small surface sample comprises at least one, and preferably two sterile swab(s) 20, at least one sterile spatula 70, in a sterile wrapper 72, at least one sterile collection tube 43, with a label 1 attached, aseptisol (aseptic) collection fluid 4, and at least one slide lock bag. All of these items are stored in a sealed bag 7 . To collect and store the sample, the pouch is opened, and the contents are removed. The sample is collected by using either a swab 20 moistened with aseptisol or with the sample is collected with the plastic spatula, particularly if it is a powder. The swab 20 or contents of the spatula 70 are deposited in the collection tube, and aseptisol is added if needed. The collection tube 43 is secured, and placed into the slide lock bag, which is then closed. The collection tube in this and in the other kits may include a label for marking the sample.

A large surface sample collection kit comprises in a pouch, at least one sterile sponge 2, in a sterile sealed whirl-pak bag 50, aseptic collection fluid 4, and at least one slide lock bag 50. It is also preferred that a sterile glove 80, preferably made out of latex and in a sterile package 81, be included in the kit.

To test a large surface for contaminants, the sealed bag 7 should be opened and the contents removed. The whirl-pak bag should be opened, and the collection fluid should be poured in, such that the sponge becomes saturated. If included, the package containing the glove should be opened, the glove removed, and worn. The sponge should be removed from the bag, and the designated sample area should be wiped. The sponge should then be returned to the bag containing the collection fluid, and the bag should be closed. This bag should then be placed into the slide lock bag, and the slide lock should be secured.

Many modifications and variations of the present invention are possible in light of the above teachings. It is, therefore, to be understood within the scope of the appended claims the invention may be protected otherwise than as specifically described.

What is claimed is:

1. A sample collection kit comprising:
at least one sterile syringe;
at least one sterile syringe filter;
at least one collection receptacle for storing the sample;
at least one container of aseptic collection fluid;
at least one bag in which the said at least one syringe, said at least one filter, said at least one collection receptacle; and said at least one container of said aseptic collection fluid are placed after being used; and
a sealed pouch for holding, prior to use, the at least one sterile syringe, said at least one sterile syringe filter, the at least one collection receptacle for storing the sample, the at least one container of aseptic collection fluid, and the at least one bag.

2. The sample collection kit of claim 1, further comprising a label for identifying the sample.

3. The sample collection kit of claim 1, further comprising a sterile package surrounding the sterile syringe.

4. The sample collection kit of claim 1, further comprising a package for the sterile filter.

5. The sample collection kit of claim 1, wherein said collection receptacle is a collection tube.

6. The sample collection kit of claim 1, wherein said aseptic collection fluid is selected from the group consisting of sterile water and sterile saline solution.

7. The sample collection kit of claim 1, wherein said at least one bag is a slide lock bag.

8. A liquid sample collection kit comprising:
at least one sterile syringe;
at least one sterile collection bag;
at least one large collection bag into which the at least one sterile collection bag and the at least one sterile syringe may be placed after said at least one sterile collection bag and said at least one sterile syringe have been used; and
a sealed pouch for holding, prior to use, the at least one sterile syringe, said at least one sterile collection bag, the at least one collection receptacle for storing the sample, the at least one container of aseptic collection fluid, and the at least one bag.

9. The liquid sample collection kit of claim 8, wherein said at least one sterile collection bag has a wire seal at a top of said collection bag running along a perimeter of the bag with extended tabs for gripping and twisting said bag closed.

10. The liquid sample collection kit of claim 8, wherein said at least one large collection bag is a slide lock bag.

11. AN air sample collection kit comprising:
at least one sterile collection tube;
at least one container of aseptic collection fluid;
at least one forceps;
at least one large collection receptacle into which the at least one sterile collection tube, the at least one container of aseptic collection, and the at least one forceps may be placed after testing is collection is completed; and
a sealed pouch for holding, prior to use, the at least one sterile collection tube, said at least one container of one aseptic collection fluid, said at least one forceps, and said at least one collection receptacle for storing the sample.

12. The air sample collection kit of claim 11 wherein said at least one large collection receptacle is a slide lock bag.

13. The air sample collection kit of claim 11, wherein said at least one sterile collection tube further comprises a label for marking said sample.

15. A collection kit for collecting a powder or a sample from a small surface, comprising:
at least one sterile swab;
at least one sterile spatula;
at least one sterile collection tube;
a container of aseptic collection fluid;
at least one slide lock bag; and
a sealed pouch for holding, prior to use, the at least one sterile swab, the at least one sterile spatula, the at least one sterile collection tube; the at least one container pf said aseptic collection fluid, and the said at least one slide lock bag.

16. The collection tube in this and in the other kits may include a label for marking the sample.
